(19) United States
(12) Patent Application Publication
(10) Pub. No.: US 2010/0234872 A1
(43) Pub. Date: Sep. 16, 2010

(54) ELECTRICAL CONTACT FOR OCCLUSIVE DEVICE DELIVERY SYSTEM

(75) Inventors: Lantao Guo, Draper, UT (US); Tra Ngo, San Jose, CA (US); Jimmy Dao, San Jose, CA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC., Maple Grove, MN (US)

(21) Appl. No.: 12/720,965

(22) Filed: Mar. 10, 2010

Related U.S. Application Data
(60) Provisional application No. 61/160,166, filed on Mar. 13, 2009.

(51) Int. Cl. A61M 29/00

(52) U.S. Cl. 606/191

(57) ABSTRACT

An occlusive coil delivery system includes a delivery wire having a distal end coupled to an occlusive device via an electrolytically severable junction, and an electrical contact secured to a proximal end of the delivery wire, which has a non-linear configuration so as to strengthen a mechanical connection with the electrical contact and/or increase electrical conductivity with the electrical contact over a linear configuration. The configuration may be, by way of non-limiting examples, a “U” shape, a spiral, a knot, or a twisted wire. The electrical contact is a conductive material that substantially envelopes the proximal end configuration of the delivery wire.

### RELATED APPLICATION DATA

The present application claims the benefit under 35 U.S.C. §119 to U.S. Provisional application Ser. No. 61/160,166 filed Mar. 13, 2009. The foregoing application is hereby incorporated by reference into the present application in its entirety.

### FIELD OF THE INVENTION

The field of the invention generally relates to systems and delivery devices for implanting vaso-occlusive devices for establishing an embolus or vascular occlusion in a vessel of a human or veterinary patient.

### BACKGROUND OF THE INVENTION

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The relative stiffness of the coil will depend, among other things, on its composition, the diameter of the wire strand, the diameter of the primary mandrel, and the pitch of the resulting primary windings. The coil is then wrapped around a larger, “secondary” mandrel, and heat treated to impart a secondary shape. For example, U.S. Pat. No. 4,994,069, issued to Ritchart et al., describes a vaso-occlusive coil that assumes a linear, helical primary shape when stretched for placement through the lumen of a delivery catheter, and a folded, convoluted secondary shape when released from the delivery catheter and deposited in the vasculature.

In order to deliver the vaso-occlusive coils to a desired site in the vasculature, e.g., within an aneurismal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive coil(s) into the aneurysm once the guidewire is withdrawn. A delivery or “pusher” wire is then passed through the micro-catheter, until a vaso-occlusive coil coupled to a distal end of the pusher wire is extended out of the distal end opening of the micro-catheter and into the aneurysm. The vaso-occlusive device is then released or “detached” from the end pusher wire, and the pusher wire is withdrawn back through the catheter. Depending on the particular needs of the patient, one or more additional occlusive devices may be pushed through the catheter and released at the same site.

One well-known way to release a vaso-occlusive coil from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes a circuit with an electrode attached to the patient's skin, or with a conductive needle inserted through the skin at a remote site, and the detachment zone disintegrates due to electrolysis.

Perceived problems with current embolic detachment schemes include mechanical weakness at the junction between the electrical contact and the delivery wire. For example, the delivery wire can be pulled out of the electrical contact during a procedure. Another perceived problem is conductive instability at the junction between the electrical contact and the delivery wire. For example, the relatively small amount of contact between the electrical contact and the delivery wire can result in less than optimal conductivity and variability in conductivity, which can lead to variability in detachment times.

Another perceived problem with some current embolic detachment devices is that a separate return or ground electrode is used to complete the electrical circuit between the external power supply and the electrolytically detachable coil. This separate return or ground electrode may be a patch that is placed on the patient's body or a needle that is inserted into the patient's groin area. The use of a separate, return or ground electrode does, however, introduce variability into the detachment time(s) of the occlusive coils. Variability is produced because of different tissue types and densities that exist between the occlusive device and the return electrode. Also, for grounding needles that are placed in the groin area of the patient, some patients experience discomfort or pain.

### SUMMARY

Embodiments of the present invention provide improved mechanical stability at the junction between the electrical contact and the delivery wire, while still providing for consistent detachment of embolic elements in the desired location. Embodiments of the present invention also reduce variability in detachment times for occlusive devices, providing conductive stability and alternative return and/or ground electrode configurations that do not utilize a separate, external return electrode, such as a patch or grounding needle.

In one embodiment, an occlusive device delivery system includes a delivery wire having a distal end coupled to an occlusive device via an electrolytically severable junction, and an electrical contact secured to a proximal end of the delivery wire, which has a non-linear configuration so as to strengthen a mechanical connection with the electrical contact and/or increase electrical conductivity with the electrical contact over a linear configuration. The configuration may be, by way of non-limiting examples, a “U” shape, a spiral, a knot, or a twisted wire. The electrical contact is a conductive material that substantially envelopes the proximal end configuration of the delivery wire. In one embodiment, the occlusive device delivery system includes a delivery wire assembly that has a proximal opening through which the proximal end of the delivery wire extends. In that embodiment, the configuration of the proximal end of the delivery wire is larger than the proximal opening so as to prevent the proximal end of the delivery wire from passing there through.

In another embodiment, a delivery wire assembly for delivery of occlusive devices to locations in a patient's vasculature includes a delivery wire conduit that has a proximal tubular portion connected to a distal coil portion, and a conduit lumen extending through the proximal tubular portion and the distal coil portion. The delivery wire assembly also includes a delivery wire disposed in the conduit lumen and having a distal end coupled to an occlusive device via an electrolytically severable junction. In addition, the delivery wire assembly includes a first electrical contact secured to a proximal end of the delivery wire, which has a non-linear configuration so as to strengthen a mechanical connection with the electrical contact. In this embodiment, a first conductive path is formed by the delivery wire, and a second conductive path is formed by the delivery wire conduit. The delivery wire assembly may also include a second electrical contact disposed on the proximal tubular portion of the delivery wire conduit, where the first and second electrical contacts are electrically coupled to the first and second conductive paths, respectively. The second electrical contact may include an exposed region of the proximal tubular portion of the delivery wire conduit. In one embodiment, the delivery wire may form a cathode and the delivery wire conduit may form an anode of a circuit formed to sever the electrolytic junction. In another embodiment, the tubular portion of the delivery wire conduit has a proximal opening, and the configuration of the proximal end of the delivery wire is larger than the proximal opening so as to prevent the proximal end of the delivery wire from passing there through. In yet another embodiment, the electrical contact is a conductive material that substantially envelopes the proximal end configuration of the delivery wire.

In still another embodiment, an occlusive coil delivery system includes a delivery catheter having a proximal end, a distal end, and a catheter lumen extending between the proximal and distal ends. The occlusive coil delivery system also includes a delivery wire assembly having a delivery wire conduit that in turn has a proximal tubular portion connected to a distal coil portion, and a conduit lumen extending through the proximal tubular portion and the distal coil portion. The delivery wire assembly also has a delivery wire disposed in the conduit lumen and having a distal end coupled to an occlusive device via an electrolytically severable junction. Further, the delivery wire assembly has a first electrical contact secured to a proximal end of the delivery wire, which has a non-linear configuration so as to strengthen a mechanical connection with the electrical contact, where a first conductive path is formed by the delivery wire, and a second conductive path is formed by the delivery wire conduit. In addition, the occlusive coil delivery system includes a power supply electrically connected to the respective first and second conductive paths. In one embodiment, the occlusive coil delivery system also includes a second electrical contact disposed on the proximal tubular portion of the delivery wire conduit, where the first and second electrical contacts are electrically coupled to the first and second conductive paths, respectively, and where the respective electrical contacts are configured to engage corresponding electrical contacts disposed in the power supply. In another embodiment, the second electrical contact includes an exposed region of the proximal tubular portion of the delivery wire conduit. In yet another embodiment, the tubular portion of the delivery wire conduit has a proximal opening, and the configuration of the proximal end of the delivery wire is larger than the proximal opening so as to prevent the proximal end of the delivery wire from passing there through. In still another embodiment, the electrical contact is a conductive material that substantially envelopes the proximal end configuration of the delivery wire.

### BRIEF DESCRIPTION OF THE DRAWINGS

Referring now to the drawings in which like reference numbers represent corresponding parts throughout, and in which:

FIG. 1 illustrates an occlusive coil delivery system, according to one embodiment.

FIG. 2A to 2D are detailed perspective views of exemplary delivery wire configurations, according to various embodiments.

FIG. 3 illustrates a cross-sectional view of a delivery wire assembly, according to one embodiment.

FIG. 4 illustrates an occlusive coil in a natural state mode, illustrating one exemplary secondary configuration.

### DETAILED DESCRIPTION OF THE ILLUSTRATED EMBODIMENTS

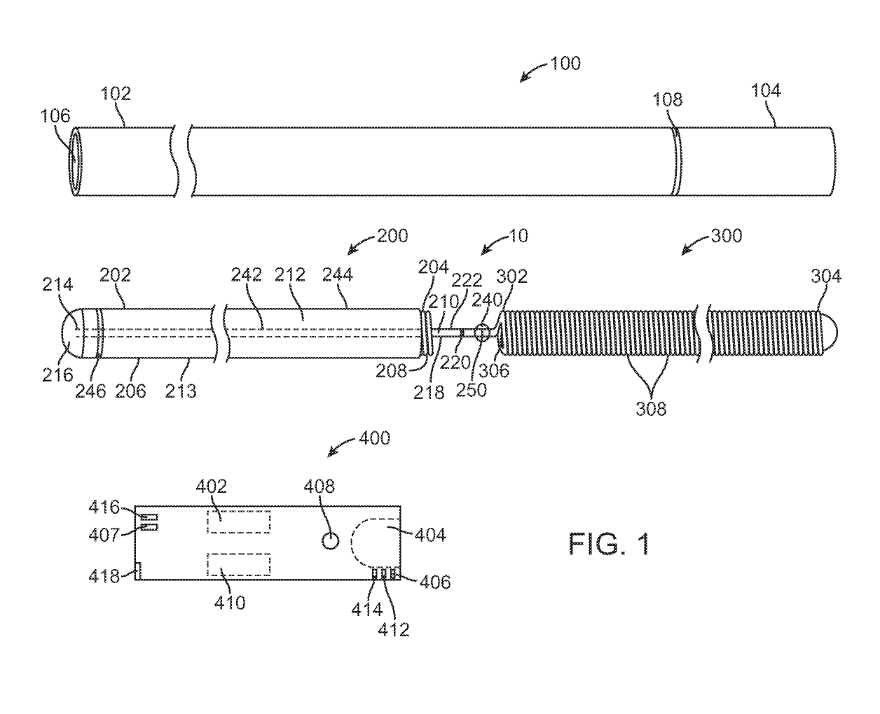

FIG. 1 illustrates an occlusive coil delivery system 10 according to one embodiment. The system 10 includes a number of subcomponents or sub-systems. These include a delivery catheter 100, a delivery wire assembly 200, an occlusive coil 300, and a power supply 400. The delivery catheter 100 includes a proximal end 102, a distal end 104, and a lumen 106 extending between the proximal and distal ends 102, 104. The lumen 106 of the delivery catheter 100 is sized to accommodate axial movement of the delivery wire assembly 200. Further, the lumen 106 is sized for the passage of a guidewire (not shown) which may optionally be used to properly guide the delivery catheter 100 to the appropriate delivery site.

The delivery catheter 100 may include a braided-shaft construction of stainless steel flat wire that is encapsulated or surrounded by a polymer coating. For example, HYDROLENE® is one exemplary polymer coating that may be used to cover the exterior portion of the delivery catheter 100. Of course, the system 10 is not limited to a particular construction or type of delivery catheter 100 and other constructions known to those skilled in the art may be used for the delivery catheter 100.

The inner lumen 106 is advantageously coated with a lubricious coating such as PTFE to reduce frictional forces between the delivery catheter 100 and the device that is being moved axially within the lumen 106. The delivery catheter 100 may include one or more optional marker bands 108 formed from a radiopaque material that can be used to identify the location of the delivery catheter 100 within the patient's vasculature system using imaging technology (e.g., fluoroscope imaging). The length of the delivery catheter 100 may vary depending on the particular application but generally is around 150 cm in length. Of course, other lengths of the delivery catheter 100 may be used with the system 10 described herein.

The delivery catheter 100 may include a distal end 104 that is straight as illustrated in FIG. 1. Alternatively, the distal end 106 may be pre-shaped into a specific geometry or orientation. For example, the distal end 104 may be shaped into a “C” shape, an “S” shape, a “J” shape, a 45° bend, a 90° bend. The size of the lumen 106 may vary depending on the size of the delivery wire assembly 200 and occlusive coil 300 but generally the diameter lumen 106 of the delivery catheter 100 (I.D. of delivery catheter 100) is less than about 0.02 inches. In some embodiments, the delivery catheter 100 may be known to those skilled in the art as a microcatheter. While not illustrated in FIG. 1, the delivery catheter 100 may be utilized with a separate guide catheter (not shown) that aids in guiding the delivery catheter 100 to the appropriate location within the patient's vasculature.

Still referring to FIG. 1, the system 10 includes a delivery wire assembly 200 that is configured for axial movement within the lumen 106 of the delivery catheter 100. The delivery wire assembly 200 generally includes a proximal end 202 and a distal end 204. In one embodiment, the delivery wire assembly 200 includes a delivery wire conduit 201, which has a proximal tubular portion 206 and a distal coil portion 208. The proximal tubular portion 206 of the delivery wire conduit 201 has a proximal opening 215 at the proximal end. The proximal tubular portion 206 may be formed from, for example, stainless steel hypotube. As explained in further detail herein, the distal coil portion 208 may be bonded to the proximal tubular portion 206 in an end-to-end arrangement. The delivery wire assembly 200 further includes a delivery wire 210 that extends from the proximal end 202 of the delivery wire assembly 200 to a location that is distal with respect to the distal end 204 of the delivery wire assembly 200. The delivery wire 210 is disposed within a lumen 212 that extends within an interior portion of the delivery wire conduit 213.

The delivery wire 210 is formed from an electrically conductive material such as stainless steel wire. The proximal end 214 of the delivery wire 210 (shown in phantom) is electrically coupled to an electrical contact 216 located at the proximal end 202 of the delivery wire assembly 200. The electrical contact 216 may be formed from a metallic solder (e.g., gold) that is configured to interface with a corresponding electrical contact (not shown) in the power supply 400.

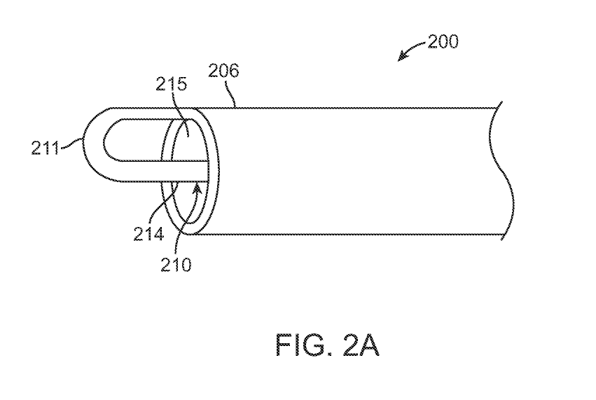

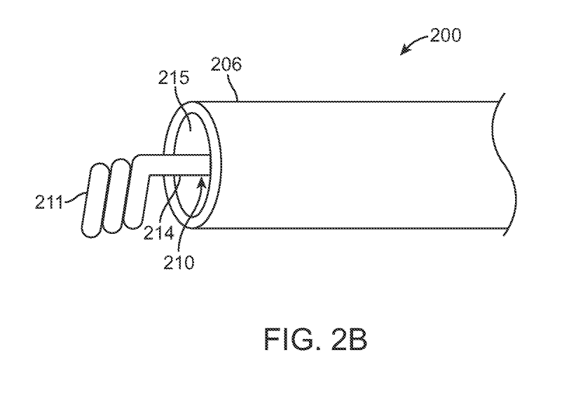

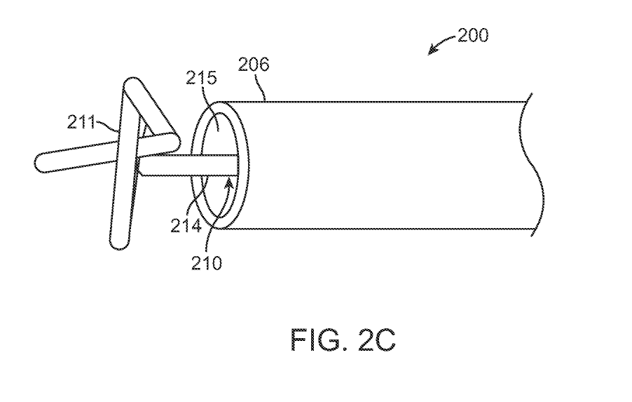

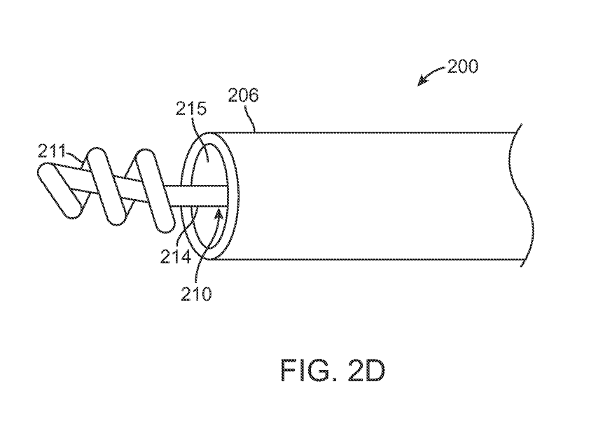

As shown in FIGS. 2A to 2D, the proximal end 214 of the delivery wire 210 takes on various configurations 211 inside of the metallic solder. Exemplary configurations 211 include a “U” shape (FIG. 2A), a spiral (FIG. 2B), a knot (FIG. 2C), and a twisted wire (FIG. 2D). Configurations 211, like a knot (FIG. 2C), may have an outer diameter (OD) larger than the inner diameter (ID) of the proximal opening 215 of the proximal tubular portion 206 of the delivery wire conduit 201. The relative sizes of the configuration 211 of the proximal end 214 of the delivery wire 210 and the proximal opening 215 prevent distal movement of the delivery wire 210 out of the delivery wire assembly 200 and the electrical contact 216. The various configurations 211 of the proximal end 214 of the delivery wire 210 also increase the amount of contact between the proximal end 214 of the delivery wire 210 and the electrical contact 216, increasing the mechanical and conductive stability of the junction between the electrical contact 216 and the delivery wire 210. The increased amount of contact between the proximal end 214 of the delivery wire 210 and the electrical contact 216 also increases the conductivity of the junction between the electrical contact 216 and the delivery wire 210.

A portion of the delivery wire 210 is advantageously coated with an insulative coating 218. The insulative coating 218 may include polyimide. In one embodiment, the entire length of the delivery wire 210 is coated with an insulative coating 218 except for the proximal end 214 of the delivery wire 210 that is in contact with electrical contact 216 and a small region 220 located in a portion of the delivery wire 210 that extends distally with respect to the distal end 204 of the of the delivery wire assembly 200. This latter “bare” portion of the delivery wire 210 forms the electrolytic detachment zone 220 which dissolves upon application of electrical current from the power supply 400.

In an alternative embodiment, instead of an electrolytic detachment zone 220, the sacrificial region may be configured to break or dissolve in response to thermal energy. For example, the detachment zone 220 may be formed from a polymeric link (e.g., fiber(s)) that melts or dissolves in response to externally applied thermal energy or heat. The polymeric link may be formed from a thermoplastic material (e.g., polyethylene) that has a high tensile strength and appropriate melting temperature. The thermally responsive sacrificial region may be responsive to an electrical resistance heater coil that is configured to apply heat to the detachment zone 220. Such heater coils operate by generating heat in response to an applied electrical current. Alternatively, electromagnetic or RF energy may be used to break or dissolve the sacrificial region. U.S. Pat. No. 7,198,613, which is incorporated herein by reference, discloses additional details regarding various thermally-actuated detachment modalities.

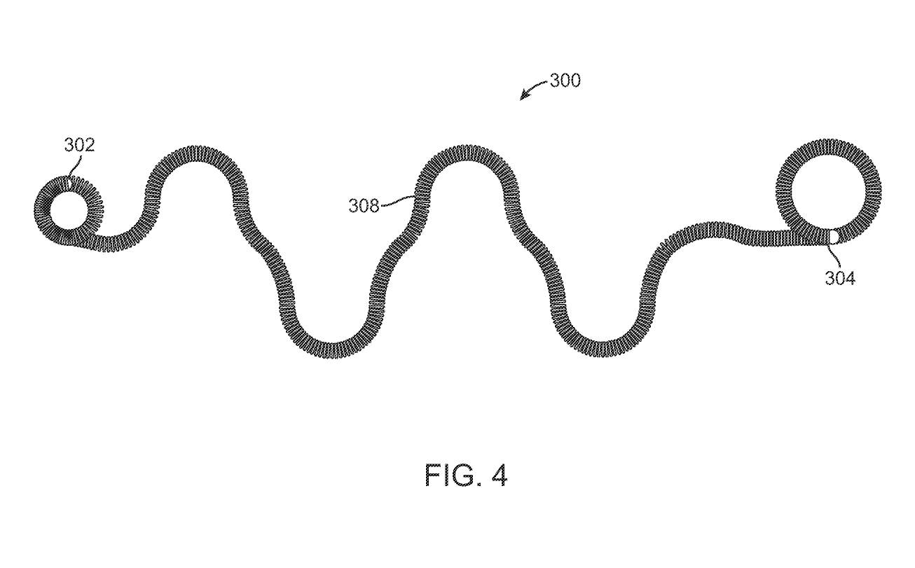

Still referring to FIG. 1, the occlusive coil 300 includes a proximal end 302, a distal end 304 and a lumen 306 extending there between. The occlusive coil 300 is generally made from a biocompatible metal such as platinum or a platinum alloy (e.g., platinum-tungsten alloy). The occlusive coil 300 generally includes a straight configuration (as illustrated in FIG. 1) when the occlusive coil 300 is loaded within the delivery catheter 100. Upon release, the occlusive coil 300 generally takes a secondary shape which may include two-dimensional or three-dimensional configurations such as that illustrated in FIG. 4. Of course, the system 10 described herein may be used with occlusive coils 300 having a variety of configurations and is not limited to particular occlusive coils 300 having a certain size or configuration.

The occlusive coil 300 includes a plurality of coil windings 308. The coil windings 308 are generally helical about a central axis disposed along the lumen 306 of the occlusive coil 300. The occlusive coil 300 may have a closed pitch configuration as illustrated in FIG. 1.

The distal end 222 of the delivery wire 210 is connected to the proximal end 302 of the occlusive coil 300 at a junction 250. Various techniques and devices can be used to connect the delivery wire 210 to the occlusive coil 300, including laser melting, and laser tack, spot, and continuous welding. It is preferable to apply an adhesive 240 to cover the junction 250 formed between the distal end 222 of the delivery wire 210 and the proximal end 302 of the occlusion coil 300. The adhesive 240 may include an epoxy material which is cured or hardened through the application of heat or UV radiation. For example, the adhesive 240 may include a thermally cured, two-part epoxy such as EPO-TEK® 353ND-4 available from Epoxy Technology, Inc., 14 Fortune Drive, Billerica, Mass. The adhesive 240 encapsulates the junction 250 and increases its mechanical stability.

Still referring to FIG. 1, the proximal tubular portion 206 and the distal coil portion 208 form a return electrode for the delivery system 10. In this regard, the delivery wire 210 forms a first conductive path 242 between the electrical contact 216 and the electrolytic detachment zone 220. This first conductive path 242 may comprise the cathode (−) of the electrolytic circuit when the delivery wire assembly 200 is operatively coupled to the power supply 400. A second conductive path 244 is formed by the proximal tubular portion 206 and a distal coil portion 208 of the delivery wire conduit 213. The second conductive path 244 is electrically isolated from the first conductive path 242. The second conductive path 244 may comprise the anode (+) or ground electrode for the electrical circuit.

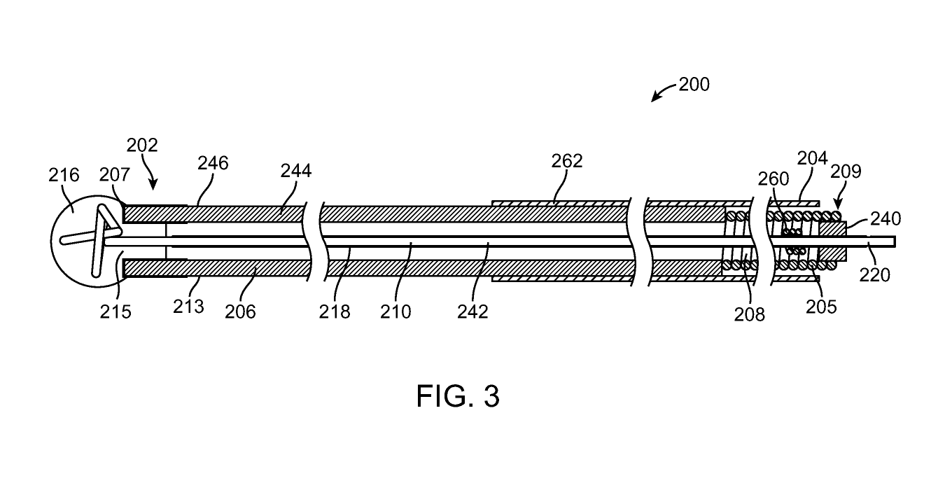

An electrical contact 246 for the second conductive path 244 may be disposed on a proximal end of the tubular portion 206 of the delivery wire conduit 213. In one embodiment, the electrical contact 246 is simply an exposed portion of the tubular portion 206 since the tubular portion 206 is part of the second conductive path 244. For instance, a proximal portion of the tubular portion 206 that is adjacent to the electrical contact 216 may be covered with an insulative coating 207 such as polyimide as illustrated in FIG. 3. An exposed region of the tubular portion 206 that does not have the insulative coating may form the electrical contact 246. Alternatively, the electrical contact 246 may be a ring type electrode or other contact that is formed on the exterior of the tubular portion 206.

The electrical contact 246 is configured to interface with a corresponding electrical contact (not shown) in the power supply 400 when the proximal end 202 of the delivery wire assembly 200 is inserted into the power supply 400. The electrical contact 246 of the second conductive path 244 is, of course, electrically isolated with respect to the electrical contact 216 of the first conductive path 242.

Still referring to FIG. 1, the system 10 includes a power supply 400 for supplying direct current to the delivery wire 210 which contains the electrolytic detachment zone 220. In the presence of an electrically conductive fluid (which may include a physiological fluid such as blood or a flushing solution such as saline), when the power supply 400 is activated, electrical current flows in a circuit including the first conductive path 242 and the second conductive path 244. After several seconds (generally less than about 10 seconds), the sacrificial electrolytic detachment zone 220 dissolves and the occlusive coil 300 separates form the delivery wire 210.

The power supply 400 will include an onboard energy source such as batteries (e.g., a pair of AAA batteries) along with drive circuitry 402. The drive circuitry 402 may include one or more microcontrollers or processors configured to output a driving current. The power supply 400 illustrated in FIG. 1 includes a receptacle 404 that is configured to receive and mate with the proximal end 202 of the delivery wire assembly 200. Upon insertion of the proximal end 202 into the receptacle 404, the electrical contacts 216, 246 disposed on the delivery wire assembly 200 electrically couple with corresponding contacts (not shown) located in the power supply 400.

A visual indicator 406 (e.g., LED light) may indicate when the proximal end 202 of delivery wire assembly 200 has been properly inserted into the power supply 400. Another visual indicator 407 may activate if the batteries need to be replaced. The power supply 400 typically includes an activation trigger or button 408 that is depressed by the user to apply the electrical current to the sacrificial electrolytic detachment zone 220. Typically, once the activation trigger 408 has been activated, the driver circuitry 402 automatically supplies current until detachment occurs. The drive circuitry 402 typically operates by applying a substantially constant current (e.g., around 1.5 mA).

The power supply 400 may include optional detection circuitry 410 that is configured to detect when the occlusive coil 300 has detached from the delivery wire 210. The detection circuitry 410 may identify detachment based upon a measured impedance value. A visual indicator 412 may indicate when the power supply 400 is being supplied to the current to the sacrificial electrolytic detachment zone 220. Another visual indicator 414 may indicate when the occlusive coil 300 has detached from the delivery wire 210. As an alternative to the visual indicator 414, an audible signal (e.g., beep) or even tactile signal (e.g., vibration or buzzer) may be triggered upon detachment. The detection circuitry 410 may be configured to disable the drive circuitry 402 upon sensing detachment of the occlusive coil 300.

The power supply 400 may also contain another visual indicator 416 that indicates to the operator when a legacy, non-bipolar delivery wire assembly is inserted into the power supply 400. As explained in the background above, prior devices used a separate return electrode that typically was in the form of a needle that was inserted into the groin area of the patient. The power supply 400 is configured to detect when one of the older non-bipolar delivery wire assemblies has been inserted. Under such situations, the visual indicator 416 (e.g., LED) is turned on and the user is advised to insert the separate return electrode (not shown in FIG. 1) into a port 418 located on the power supply 400.

FIG. 3 illustrates a cross-sectional view of the delivery wire assembly 200 according to one embodiment. Similar elements of this embodiment are identified with the same reference numbers as discussed above with respect to FIGS. 1 and 2A to 2D. The delivery wire assembly 200 includes a proximal end 202 and a distal end 204 and measures between around 183 cm to around 187 cm in length. The delivery wire assembly 200 includes a delivery wire conduit 213 with a proximal tubular portion 206 and a distal coil portion 208. The proximal tubular portion 206 may be formed from stainless steel hypotube having an OD of 0.0125 inches and ID of 0.00825 inches. The length of the hypotube section may be between around 140 cm to around 150 cm, although other lengths may also be used.

As seen in FIG. 3, a distal coil portion 208 is bonded in end-to-end fashion to the distal face of the proximal tubular portion 206. The bonding may be accomplished using a weld or other bond. The distal coil portion 208 may have a length of around 39 cm to around 41 cm in length. The distal coil portion 208 may comprise a coil of 0.0025 inches×0.006 inches. This dimension generally refers to the internal mandrel used to wind the coil wire around to form the plurality of coil winds and is the nominal ID of the coil.

One or more coils 205 of the distal coil portion 208 may be formed from a radiopaque material (illustrated as solid coils 205 in distal coil portion 208). For example, the distal coil portion 208 may include a segment of stainless steel coil (e.g., 3 mm in length), followed by a segment of platinum coil (which is radiopaque and also 3 cm in length), followed by a segment of stainless steel coil (e.g., 3 mm in length), and so on and so forth.

A delivery wire 210 forms the first conductive path 242 and terminates at electrical contact 216 at one end and extends distally with respect to the distal coil portion 208 of the delivery wire conduit 213. The delivery wire 210 is coated with an insulative coating 218 such as polyimide except at the electrolytic detachment zone 220 and the proximal segment coupled to the electrical contact 216. The delivery wire 210 may have an OD of around 0.0125 inches. A centering coil 260 is affixed to the delivery wire 210 at a location within the distal coil portion 208. The centering coil 260 ensures that the delivery wire 210 is properly oriented within the delivery wire assembly 200. The centering coil 260 may be bonded directly to the delivery wire 210 using an adhesive 240 such as that described herein. To this end, an adhesive 240 is applied to secure the delivery wire 210 and centering coil 260 to the distal coil portion 208. The adhesive 240 may include EPO-TEK® 353ND-4 described in more detail above.

Still referring to FIG. 3, an outer sleeve 262 or jacket surrounds a portion of the proximal tubular portion 206 and a portion of the distal coil portion 208 of the delivery wire conduit 213. The outer sleeve 262 covers the interface or joint formed between the proximal tubular portion 206 and the distal coil portion 208. The outer sleeve 262 may have a length of around 50 cm to around 54 cm. The outer sleeve 262 may be formed from a polyether block amide plastic material (e.g., PEBAX 7233 lamination). The outer sleeve 262 may include a lamination of PEBAX and HYDROLENE®. The OD of the outer sleeve 262 may be less than 0.02 inches and advantageously less than 0.015 inches.

As seen in FIG. 3, a small segment 209 of the distal coil portion 208 is exposed distally beyond the outer sleeve 262. During use, this small segment 209 is exposed to conductive fluids and serves as the contact for the second conductive path 244 (e.g., return or ground path) of the circuit. This segment that projects distally may have a length greater than about 0.03 inches. The electrolytic detachment zone 220 is located several centimeters (e.g., about 2 to about 4 cm) distally with respect to the distal end of the distal coil portion 208.

FIG. 4 illustrates one exemplary configuration of an occlusive coil 300 in a natural state. In the natural state, the occlusive coil 300 transforms from the straight configuration illustrated in, for instance, FIG. 1 into a secondary shape. The secondary shaped may include both two and three dimensional shapes of a wide variety. FIG. 4 is just one example of a secondary shape of an occlusive coil 300 and other shapes and configurations are contemplated to fall within the scope of the invention. Also, the occlusive coil 300 may incorporate synthetic fibers over all or a portion of the occlusive coil 300 as is known in the art. These fibers may be attached directly to coil windings 308 or the fibers may be integrated into the occlusive coil 300 using a weave or braided configuration.

The configurations 211 of the proximal end 214 of the delivery wire 210 provide a number of advantages over previous embolic coil delivery systems. First the configurations 211 increase the mechanical stability of the connection between the delivery wire 210 and the electrical contact 216. The combination of a proximal opening 215 and a configuration 211 with an OD larger than the ID of the proximal opening 215 further increases mechanical stability. The configurations 211 also increase conductive stability of the connection between the delivery wire 210 and the electrical contact 216, by increasing the mechanical stability and by increasing the amount of contact between the delivery wire 210 and the electrical contact 216. The increase in amount of contact also increases conductivity between the delivery wire 210 and the electrical contact 216.

Another benefit of the system 10 described herein is that it utilizes a bipolar arrangement of the conductive paths 242, 244 in the actual delivery wire assembly 200. There is no longer any need to use a separate needle electrode that is inserted into the patient's groin area. Instead, the return or ground electrode is integrated into delivery wire assembly 200. This not only eliminates the need for the needle electrode but it results in more reproducible detachment times because there is no longer a large volume of tissue existing through which electrical current must pass.

The electrical contact 216 may be manufactured by inserting a delivery wire 210 into the lumen 212 of the delivery wire conduit 213. Then the proximal end 214 of the delivery wire 210 may be formed into a three dimensional configuration 211. A metallic solder can then be applied to the proximal end 202 of the delivery wire assembly 200, covering the configuration 211 and forming the electrical contact 216. After the metallic solder is allowed to cure, clippers or the like may be used to trim the excess material.

While various embodiments of the present invention have been shown and described, they are presented for purposes of illustration, and not limitation. Various modifications may be made to the illustrated and described embodiments without departing from the scope of the present invention, which is to be limited and defined only by the following claims and their equivalents.

What is claimed is:

1. An occlusive device delivery system, comprising:
a delivery wire having a distal end coupled to an occlusive device via an electrolytically severable junction; and
an electrical contact secured to a proximal end of the delivery wire, the proximal end of the delivery wire having a non-linear configuration so as to strengthen a mechanical connection with the electrical contact.

2. The occlusive device delivery system of claim 1, wherein the configuration of the proximal end of the delivery wire creates an increase in electrical conductivity with the electrical contact over a linear configuration.

3. The occlusive device delivery system of claim 1, wherein the configuration of the proximal end of the delivery wire is a three dimensional shape.

4. The occlusive device delivery system of claim 1, wherein the configuration of the proximal end of the delivery wire is a “U” shape.

5. The occlusive device delivery system of claim 1, wherein the configuration of the proximal end of the delivery wire is a spiral.

6. The occlusive device delivery system of claim 1, wherein the configuration of the proximal end of the delivery wire is a knot.

7. The occlusive device delivery system of claim 1, wherein the configuration of the proximal end of the delivery wire is a twisted wire.

8. The occlusive device delivery system of claim 1, wherein the electrical contact comprises a conductive material that substantially envelopes the proximal end configuration of the delivery wire.

9. The occlusive device delivery system of claim 1, further comprising a delivery wire assembly having a proximal opening through which the proximal end of the delivery wire extends, wherein the configuration of the proximal end of the delivery wire is larger than the proximal opening so as to prevent the proximal end of the delivery wire from passing there through.

10. A delivery wire assembly for delivery of occlusive devices to locations in a patient's vasculature, comprising:
a delivery wire conduit having a proximal tubular portion connected to a distal coil portion, and a conduit lumen extending through the proximal tubular portion and the distal coil portion;
a delivery wire disposed in the conduit lumen and having a distal end coupled to an occlusive device via an electrolytically severable junction; and
a first electrical contact secured to a proximal end of the delivery wire, the proximal end of the delivery wire having a non-linear configuration so as to strengthen a mechanical connection with the electrical contact,
wherein a first conductive path is formed by the delivery wire, and a second conductive path is formed by the delivery wire conduit.

11. The delivery wire assembly of claim 10, further comprising a second electrical contact disposed on the proximal tubular portion of the delivery wire conduit, wherein the first and second electrical contacts are electrically coupled to the first and second conductive paths, respectively.

12. The delivery wire assembly of claim 11, wherein the second electrical contact comprises an exposed region of the proximal tubular portion of the delivery wire conduit.

13. The delivery wire assembly of claim 10, wherein the delivery wire comprises a cathode and the delivery wire conduit comprises an anode of a circuit formed to sever the electrolytic junction.

14. The delivery wire assembly of claim 10, the tubular portion of the delivery wire conduit comprising a proximal opening, wherein the configuration of the proximal end of the delivery wire is larger than the proximal opening so as to prevent the proximal end of the delivery wire from passing there through.

15. The delivery wire assembly of claim 10, wherein the first electrical contact comprises a conductive material that substantially envelopes the proximal end configuration of the delivery wire.

16. An occlusive coil delivery system, comprising:
a delivery catheter comprising a proximal end, a distal end, and a catheter lumen extending between the proximal and distal ends;
a delivery wire assembly comprising
a delivery wire conduit having a proximal tubular portion connected to a distal coil portion, and a conduit lumen extending through the proximal tubular portion and the distal coil portion,
a delivery wire disposed in the conduit lumen and having a distal end coupled to an occlusive device via an electrolytically severable junction,
a first electrical contact secured to a proximal end of the delivery wire, the proximal end of the delivery wire having a non-linear configuration so as to strengthen a mechanical connection with the electrical contact, wherein a first conductive path is formed by the delivery wire, and a second conductive path is formed by the delivery wire conduit; and
a power supply electrically connected to the respective first and second conductive paths.

17. The system of claim 16, further comprising a second electrical contact disposed on the proximal tubular portion of the delivery wire conduit, wherein the first and second electrical contacts are electrically coupled to the first and second conductive paths, respectively, and wherein the respective electrical contacts are configured to engage corresponding electrical contacts disposed in the power supply.

18. The system of claim 17, wherein the second electrical contact comprises an exposed region of the proximal tubular portion of the delivery wire conduit.

19. The system of claim 16, the tubular portion of the delivery wire conduit comprising a proximal opening, wherein the configuration of the proximal end of the delivery wire is larger than the proximal opening so as to prevent the proximal end of the delivery wire from passing there through.

20. The system of claim 16, wherein the first electrical contact comprises a conductive material that substantially envelopes the proximal end configuration of the delivery wire.
